(19) United States
(12) Patent Application Publication
(10) Pub. No.: US 2025/0205699 A1
(43) Pub. Date: Jun. 26, 2025

(54) Smartphone-Compatible Urine Test Strip with Integrated Calibration and Enhanced Biomarker Analysis

(71) Applicant: Vivosens Inc., San Francisco, CA (US)

(72) Inventors: Miray Tafun, San Francisco, CA (US); George Radman, San Francisco, CA (US)

(21) Appl. No.: 19/080,859

(22) Filed: Mar. 15, 2025

Related U.S. Application Data
(63) Continuation-in-part of application No. 17/052,533, filed on Nov. 3, 2020.

(30) Foreign Application Priority Data
May 15, 2018 (WO) PCT/TR2018/050233

(51) Int. Cl. B01L 3/00; G01N 21/77; G01N 21/78

(52) U.S. Cl. CPC B01L 3/5023; G01N 21/78; B01L 2300/021; B01L 2300/069; B01L 2300/0825; G01N 2021/7759; G01N 2201/127

(57) ABSTRACT

The present invention relates to a urine test strip system designed for at-home use, enabling the detection and analysis of multiple urinary biomarkers with enhanced accuracy and digital integration. The system comprises a test strip featuring a white polymer substrate, a plurality of chemically treated test pads, reference calibration markers, a machine-readable code, and a protective overlay. The reference calibration markers consist of distinct color patterns that standardize colorimetric analysis, compensating for environmental factors such as lighting variations and camera inconsistencies. The machine-readable code, such as a QR code, provides batch-specific calibration data and assists in image alignment when scanned by a companion smartphone application. The protective overlay prevents contamination while ensuring optimal fluid absorption. A dedicated smartphone application processes the captured image, identifies test pad reactions, applies color correction algorithms, and interprets biomarker levels using predefined thresholds.

### BACKGROUND OF THE INVENTION

### Field of Invention

The present invention relates to the field of medical diagnostic testing, specifically to urinalysis test strips that enable the detection and analysis of multiple biomarkers in urine samples. More particularly, the invention pertains to at-home urine test systems that utilize smartphone-based image processing for automated result interpretation, calibration, and data tracking. The invention further addresses improvements in test strip design, reference calibration, and digital integration to enhance accuracy, reliability, and ease of use in diverse environmental conditions.

### Brief Summary of the Invention

This invention relates to a urine test strip system designed for at-home use to measure and analyze multiple biomarkers in a single urine sample. The system integrates a smartphone application that captures and interprets the results, providing a more accurate and user-friendly experience compared to traditional urine testing methods. Key features of the invention include a reconfigured layout of test pads for enhanced accuracy when scanned by a smartphone camera, the inclusion of a machine-readable code for precise calibration and alignment, and a redesigned physical form factor of the strip to ensure consistent colorimetric readings. Additionally, the test strip incorporates a protective overlay to prevent contamination of the chemical pads and ensures rapid fluid absorption. These enhancements enable improved reliability in test results, reduce user error, and provide comprehensive health data through digital integration. The system is designed to evaluate multiple biomarkers at once and track results over time, offering users a more thorough analysis of their health.

### BRIEF DESCRIPTION OF THE FIGURES

FIG. 1 illustrates a urine test strip (1) designed for at-home biomarker analysis, featuring a white polymer substrate (1), chemically treated test pads (2), a machine-readable code (3), a protective overlay (4), reference calibration markers (5), a designated grip area (6), and optional edge guides or notches (7) to enhance usability, accuracy, and digital integration.

### DETAILED DESCRIPTION

The present invention relates to an advanced urine test strip system designed for at-home use, enabling the measurement and analysis of multiple biomarkers in a single test. Unlike traditional urine test strips that rely on manual color comparison, this system integrates a smartphone application for automated result interpretation, significantly improving accuracy and user experience. The invention incorporates a redesigned test strip with a structured layout of chemically treated test pads, reference calibration colors, and a machine-readable code to optimize precision, consistency, and ease of use. Through the integration of image processing and real-time calibration adjustments, the system ensures reliable colorimetric analysis regardless of variations in lighting, camera hardware, or environmental conditions.

The test strip consists of a substrate, a plurality of chemically treated test pads, a protective overlay, reference calibration markers, and a machine-readable identification code. The substrate serves as the structural foundation of the strip, providing support for all functional components. It is composed of a white polymer backing material that prevents urine from spreading uncontrollably beyond designated test areas. The substrate is designed to maintain rigidity while remaining thin enough to allow for rapid and controlled absorption of liquid.

The test pads are the core analytical components of the strip, each containing reagent chemistries specifically formulated to react with different urinary biomarkers. These pads are arranged in a structured pattern, such as a diagonal layout or a two-row format, to optimize fluid absorption, reduce cross-contamination, and facilitate precise scanning by a smartphone camera. Each test pad is spatially separated from adjacent pads to prevent unintended chemical interference, ensuring accurate reaction results. The test pads can detect a variety of analytes, including but not limited to ketones, pH levels, proteins, glucose, leukocytes, and nitrites. The number and type of test pads can be modified depending on the intended application, allowing for customization to address various health monitoring needs.

To account for environmental variability and ensure accurate colorimetric analysis, the invention incorporates reference calibration markers adjacent to the test pads. These markers consist of 24 distinct colors, arranged in a predetermined pattern, to provide a standardized reference for the smartphone application's image processing algorithms. The reference colors compensate for variations caused by different lighting conditions, shadows, camera angles, and smartphone hardware discrepancies. Additionally, the strip includes four white squares positioned at its corners, which serve as alignment markers for precise location detection within the captured image. The smartphone application utilizes these reference points to correct for tilt, distortion, or improper positioning of the strip during analysis.

A machine-readable code, such as a QR code, is positioned near the reference calibration markers to further enhance accuracy and provide additional functional benefits. This code stores batch identification data, production date information, and pre-recorded calibration values specific to that particular batch of test strips. Upon scanning the strip, the smartphone application retrieves this calibration data and applies necessary color correction algorithms to ensure accurate interpretation of test results. The QR code also assists in automatically orienting the image to the correct positioning, ensuring that the test pads are analyzed in the appropriate order. In alternative embodiments, other types of machine-readable codes, such as barcodes, DataMatrix codes, or proprietary digital markers, may be used to achieve similar calibration and alignment functions.

To preserve the integrity of the test pads and prevent contamination, the invention includes a protective overlay composed of a transparent or semi-transparent material. This layer shields the chemically treated pads from external contaminants such as moisture, dust, and physical contact while allowing urine to flow freely into the pads for analysis. The protective layer also prevents premature exposure of the reagent chemistries, ensuring consistent performance and reliable test results over time.

The handle or grip area of the strip is specifically designed to remain free of reactive chemicals, providing a designated section for users to hold the strip without interfering with test pad reactions. This design feature ensures ease of handling while minimizing the risk of contamination. Additionally, optional edge guides or notches may be integrated into the strip to assist users in aligning the strip correctly within the smartphone camera's field of view. These alignment features improve scanning consistency and reduce potential user errors.

In operation, the user immerses the test strip into a urine sample for a predetermined duration, ensuring uniform exposure of all test pads. After removing the strip, the user launches the companion smartphone application and follows guided instructions to capture an image of the strip under appropriate lighting conditions. The application then processes the image, identifying the test pads, extracting colorimetric data, and comparing the reaction colors to the reference calibration markers. Advanced image processing algorithms correct for potential distortions caused by ambient lighting, camera sensor variations, and improper positioning. The processed data is then interpreted using a database of predefined biomarker thresholds, generating a comprehensive set of test results displayed on the user's smartphone screen.

The smartphone application provides users with real-time health insights based on the detected biomarker levels. Additionally, the application stores historical test results, allowing users to track trends and monitor changes in their urinary biomarkers over time. Users may also choose to share their results with healthcare providers for further evaluation and medical advice. The system's cloud-based architecture enables secure data storage and remote access to test records, enhancing long-term health monitoring capabilities.

This invention offers several advantages over existing urine test systems, including enhanced accuracy through integrated calibration, improved user experience through guided digital analysis, and increased reliability in diverse environmental conditions. Unlike conventional urine test strips that require subjective manual interpretation, this system leverages smartphone-based automation to eliminate human error and provide precise, reproducible results. The modular design of the strip allows for future adaptations, supporting additional biomarkers and expanded health assessments. The integration of digital tracking and data analysis transforms at-home urine testing into a comprehensive, user-friendly diagnostic tool.

Furthermore, the invention incorporates a black reference area strategically positioned adjacent to the test pads, which serves to enhance contrast and improve detection accuracy by minimizing the effects of external light interference. This black background allows the smartphone application's image processing algorithms to distinguish reaction colors with higher precision, resulting in more reliable test readings.

Additionally, the smartphone application utilizes machine learning algorithms to further improve result accuracy by continuously refining its color recognition and correction processes. By analyzing patterns from past test data, the system adapts to variations in user imaging conditions, different smartphone cameras, and potential environmental inconsistencies, thereby ensuring more consistent and precise biomarker assessments.

The test strip itself is constructed from a specialized paper-based material, allowing for controlled fluid absorption while maintaining structural integrity throughout the testing process. This paper-based composition ensures optimal performance making the strip both cost-effective and environmentally friendly.

The combination of these enhancements—machine learning-driven analysis, optimized contrast through the black reference area, and the use of appropriate materials—ensures that the invention provides a highly accurate, user-friendly, and technologically advanced solution for at-home urine biomarker testing.

### DETAILED DESCRIPTION OF FIGURES

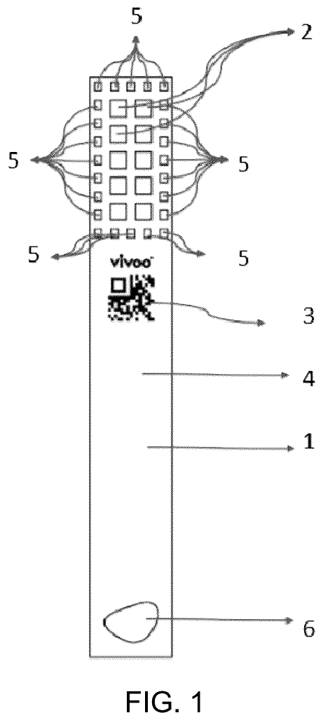

FIG. 1 depicts a urine test strip (1) comprising a white polymer substrate that provides structural integrity while preventing unintended fluid absorption beyond designated test areas. The substrate is composed of a plastic blend, ensuring rigidity while maintaining a thin profile for ease of handling.

The test strip includes multiple chemically treated test pads (2) strategically placed along its surface. Each pad is formulated to react with specific urinary biomarkers, such as ketones, pH, magnesium, calcium, creatinine, sodium, free radicals, bilirubin, urobilinogen, specific gravity, proteins, glucose, leukocytes, and nitrites. The test pads are spatially separated to prevent cross-contamination and are designed with defined square areas that correspond to reaction zones. The size and spacing of the pads are optimized for accurate colorimetric analysis when scanned by a smartphone camera.

To facilitate accurate readings and ensure proper test strip alignment, a machine-readable code (3), such as a QR code, is positioned near the reference calibration markers. This code serves multiple functions, including identifying the strip batch and lot number, storing calibration data to compensate for variations in lighting and camera hardware, and assisting the smartphone application in correctly orienting the strip within the captured image.

A protective overlay (4) is applied over the test pads, providing a transparent or semi-transparent barrier to shield the reactive areas from premature exposure or contamination. This layer ensures that the pads remain dry and uncontaminated before use while still allowing urine to wick through to the reactive areas efficiently. The overlay prevents environmental factors such as dust, moisture, or accidental handling from affecting test accuracy.

Adjacent to the test pads, the strip incorporates reference calibration markers (5) consisting of a set of distinct color squares. These reference colors provide a standardized baseline for colorimetric analysis, enabling the smartphone application to adjust for variations in ambient lighting, image distortion, and camera sensor differences. The reference colors are pre-measured using a colorimeter and assigned specific values, ensuring consistency across different batches of test strips.

For user convenience, the strip features a designated grip area (6) located at one end. This section remains free of reactive chemicals, allowing users to handle the strip without affecting test results. The grip area ensures ease of use while minimizing contamination risks.

Additionally, edge guides or notches (7) may be integrated into the strip's design to assist in proper alignment during scanning or to provide depth guidance when dipping the strip into a urine sample. These structural features help users position the strip consistently in front of the smartphone camera, improving scanning accuracy and reducing variability in test results.

What is claimed is:

1. A urine test strip system for measuring and analyzing multiple biomarkers, comprising:
a. a substrate having a paper-based composition configured to provide structural support and controlled fluid absorption;
b. a plurality of chemically treated test pads disposed on the substrate, each test pad formulated to react with a specific urinary biomarker and spaced to minimize cross-contamination;
c. a set of reference calibration markers positioned adjacent to the test pads, the reference calibration markers comprising a plurality of distinct colors configured to enable standardized colorimetric analysis;
d. a machine-readable code positioned in proximity to the reference calibration markers, the machine-readable code storing calibration data, batch identification, and strip orientation information;
e. a black reference area positioned adjacent to the test pads, the black reference area configured to enhance contrast and improve detection accuracy by minimizing external light interference;
f. a protective overlay covering at least a portion of the test pads, the protective overlay being transparent or semi-transparent and configured to prevent contamination while allowing fluid absorption; and
g. a designated handle area free of reactive chemicals, allowing a user to hold the test strip without interfering with test pad reactions.

2. The urine test strip system of claim 1, wherein the test pads are arranged in a structured layout optimized for uniform fluid absorption and accurate scanning by a smartphone camera.

3. The urine test strip system of claim 1, wherein the reference calibration markers consist of at least 24 distinct colors configured to compensate for lighting variability, image distortion, and environmental inconsistencies.

4. The urine test strip system of claim 1, wherein the machine-readable code is a QR code, DataMatrix code, or barcode and is configured to store calibration values specific to the test strip batch.

5. The urine test strip system of claim 1, wherein the machine-readable code provides orientation guidance to the smartphone application, enabling automatic alignment and correction of the captured image.

6. The urine test strip system of claim 1, wherein the protective overlay is composed of a transparent or semi-transparent material that shields the test pads from premature exposure to moisture and contaminants.

7. The urine test strip system of claim 1, wherein the test pads are capable of detecting at least one of the following urinary biomarkers: ketones, glucose, magnesium, calcium, creatinine, sodium, free radicals, bilirubin, urobilinogen, specific gravity, proteins, leukocytes, nitrites, pH, or other analytes indicative of health conditions.

8. The urine test strip system of claim 1, further comprising four white alignment markers located at the corners of the substrate, wherein the alignment markers assist in identifying the strip's position within a captured image.

9. The urine test strip system of claim 1, wherein the test strip is scanned using a smartphone application configured to:
a. capture an image of the test strip,
b. identify the test pads and reference calibration markers,
c. apply color correction algorithms using the calibration markers,
d. interpret biomarker levels based on predefined threshold values, and
e. display test results on the smartphone screen.

10. The urine test strip system of claim 9, wherein the smartphone application further provides real-time health insights, trend tracking, and the ability to share test results with healthcare providers.

11. The urine test strip system of claim 9, wherein the smartphone application retrieves calibration data from the machine-readable code to adjust colorimetric analysis based on production variations and environmental conditions.

12. The urine test strip system of claim 1, wherein the test strip includes structural features such as edge guides or notches to assist users in aligning the strip correctly within the smartphone camera's field of view.

13. A method of performing urine analysis using the urine test strip system of claim 1, the method comprising:
a. immersing the test strip into a urine sample for a predetermined duration;
b. removing the test strip and placing it in a designated scanning position;
c. capturing an image of the test strip using a smartphone application;
d. processing the image to identify the test pads, reference calibration markers, and machine-readable code;
e. adjusting the colorimetric analysis using calibration data retrieved from the machine-readable code and reference markers; and
f. displaying biomarker test results on the smartphone application.

14. The method of claim 13, wherein the smartphone application applies machine learning algorithms to enhance result accuracy by compensating for variations in lighting, camera hardware, and image quality through pattern recognition and adaptive color correction.

15. The method of claim 13, further comprising storing the test results in a digital database for trend tracking, historical analysis, and remote healthcare monitoring.

16. The method of claim 13, wherein the smartphone application provides instructional feedback to the user regarding optimal lighting conditions and camera positioning before capturing the test strip image.

17. The method of claim 13, wherein the smartphone application generates personalized health recommendations based on the detected biomarker levels.

18. The urine test strip system of claim 1, wherein the test pads and reference calibration markers are printed using a high-precision colorimetric printing process to ensure consistency across different test strip batches.

19. The urine test strip system of claim 1, wherein the test strip is disposable and designed for single-use applications.

20. The urine test strip system of claim 1, wherein the smartphone application integrates with cloud-based storage systems to enable remote access to test results and health monitoring data.
